(19) United States
(12) Patent Application Publication
(10) Pub. No.: US 2010/0317689 A1
(43) Pub. Date: Dec. 16, 2010

(54) Prodrugs of proton pump inhibitors including the 1h-imidazo[4,5-b] pyridine moiety

(76) Inventors: Michael E. Garst, Newport Beach, CA (US); George Sachs, Encino, CA (US); Jai Moo Shin, Chatsworth, CA (US)

Correspondence Address: Gabor L Szekeres;Law Offices of Gabor L Szekeres, P O Box 27938, Anaheim, CA, 92809, US

(21) Appl. No.: 12/310,867

(22) PCT Filed: Sep. 15, 2007
(86) PCT No.: PCT/US2007/019991

§ 371 Date: Oct. 28, 2009

Related U.S. Application Data
(60) Provisional application No. 60/845,666, filed on Sep. 19, 2006.

(51) Int. Cl. A61K 31/437; C07D 471/02; A61P 1/00; A61P 1/04

(52) U.S. Cl. 514/303; 546/118

(57) ABSTRACT

The compounds of formulas (I), (II), (III) and (IV), where the symbols are as defined in the specification are prodrugs of proton pump inhibitors. The R₄ group includes at least one acidic group or its pharmaceutically acceptable salt, and the compound having the R₄ group have improved aqueous solubility, stability in plasma and improved bioavailability.

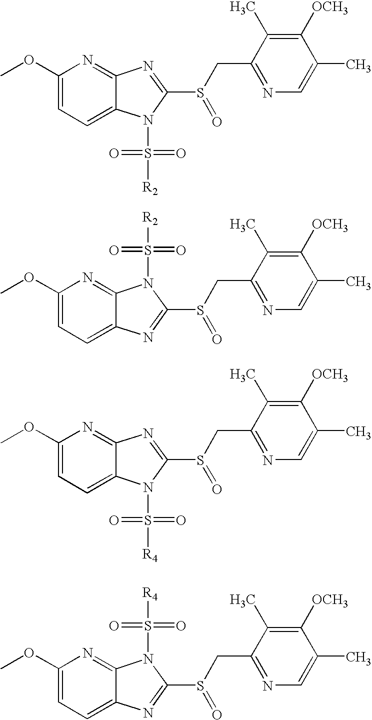

### BACKGROUND OF THE INVENTION

1. Field of the Invention

The present invention is directed to prodrugs of proton pump inhibitors which are useful as anti-ulcer agents. More particularly, the present invention is directed to prodrugs of proton pump inhibitors which include the 1H-imidazo[4,5-b]pyridine moiety. The prodrugs of the present invention slowly hydrolyze to provide the proton pump inhibitor including the above-noted structure which inhibit exogenously or endogenously gastric acid secretion and can be used in the prevention and treatment of gastrointestinal inflammatory diseases in mammals, including humans.

2. Brief Description of the Prior Art

Benzimidazole derivatives intended for inhibiting gastric acid secretion are disclosed in U.S. Pat. Nos. 4,045,563; 4,255,431; 4,628,098; 4,686,230; 4,758,579; 4,965,269; 5,021,433; 5,430,042, 5,554,631, 5,703,097, 5,708,017 and 6,599,167. An imidazo[4,5-b]pyridine derivative intended for the same purpose is disclosed in U.S. Pat. No. 4,808,596. Generally speaking, these inhibitors of gastric acid secretion work by undergoing a rearrangement to form a thiophilic species which then covalently binds to gastric H,K-ATPase, the enzyme involved in the final step of proton production in the parietal cells, and thereby inhibits the enzyme. Compounds which inhibit the gastric H,K-ATPase enzyme are generally known in the field as “proton pump inhibitors” (PPI).

Some of the benzimidazole compounds capable of inhibiting the gastric H,K-ATPase enzyme have found substantial use as drugs in human medicine and are known under such names as LANSOPRAZOLE (U.S. Pat. No. 4,628,098), OMEPRAZOLE (U.S. Pat. Nos. 4,255,431 and 5,693,818), PANTOPRAZOLE (U.S. Pat. No. 4,758,579), and RABEPRAZOLE (U.S. Pat. No. 5,045,552). The diseases treated by proton pump inhibitors and specifically by the four above-mentioned drugs include peptic ulcer, heart burn, reflux esophagitis errosive esophagitis, non-ulcer dispepsia, infection by Helicobacter pylori, laryngitis and asthma among others.

U.S. Pat. Nos. 5,554,631 and 5,703,097 disclose the proton pump inhibitor compound known by the chemical name 2-((4-methoxy-3-methylpyridin-2-yl)methylsulfinyl)-5-(1H-pyrrol-1-yl)-1H-benzo[d]imidazole (or by the alternative chemical name 2-((4-methoxy-3-methylpyridin-2-yl)methylsulfinyl)-5-(1H-pyrrol-1-yl)-1H-benzimidazole), generally known as ILAPRAZOLE. U.S. Pat. No. 4,808,596 disclose the proton pump inhibitor compound known by the chemical name 5-methoxy-2-((4-methoxy-3,5-dimethylpyridin-2-yl)methylsulfinyl)-3H-imidazo[4,5-b]pyridine, generally known as TENATOPRAZOLE (U.S. Pat. No. 4,808,596).

Whereas the proton pump inhibitor type drugs represent substantial advance in the field of human and veterinary medicine, they are not totally without shortcomings or disadvantages. The shortcomings of the presently used proton pump inhibitor (PPI) type drugs can be best explained by a more detailed description of the mode of their action, the diseases or condition against which they are employed and the circumstances of their application. Thus, acid related diseases include but are not limited to erosive esophagitis, esophageal reflux, gastric and duodenal ulcer, non-ulcer dyspepsia and infection by Helicobacter pylori. Current therapy of all but the infection by H. pylori bacteria involves treatment with drugs designed to suppress acid secretion, one type of which are the above-mentioned proton pump inhibitors.

The presently used proton pump inhibitors are pyridyl methyl sulfinyl benzimidazoles (or compounds of related structure, such as 5-methoxy-2-((4-methoxy-3,5-dimethylpyridin-2-yl)methylsulfinyl)-3H-imidazo[4,5-b]pyridine, generally known as TENATOPRAZOLE) with a stated pKₐ of 4.0 to 5.0. Their mechanism of action requires accumulation in the acidic space of the parietal cell (secretory canaliculus, pH ca. 1.0) and subsequently hydrogen ion catalyzed conversion to the reactive thiophilic species that is capable of inhibiting the gastric ATPase, enzyme resulting in effective inhibition of gastric secretion with a large therapeutic index. Because of this mechanism the presently used PPI type drugs require specialized gastro protection to remain active for duodenal absorption. For this reason, and due to sensitivity to degradation in the acid milieu of the stomach, oral formulations of the PPI drugs are enteric coated. The need for enteric coating is a shortcoming because enteric coating is expensive and moisture sensitive.

Because of the requirement for accumulation in the acid space of the parietal cell, acid secretion is necessary for the efficacy of the PPI type drugs. It was found that the plasma half life of these drugs is between 60 to 90 minutes. All acid pumps are not active at any one time, rather only about 75% are active on the average during the time the drug is present in the blood following oral administration. It was also found in medical experience that on a currently used once-a-day oral administration therapy the maximal inhibition of stimulated acid output is approximately 66%. This is due to a combination of the short plasma half life of the drug, to the limited number of acid pumps active during presentation of the drug and to the turn-over of acid pumps. In present practice it is often not possible to properly control night time acid secretion by evening therapy with oral administration because the drug is dissipated from the plasma by the time acid secretion is established after midnight. The ideal target for healing in acid related diseases and for treatment of H. pylori infection (in conjunction with antibiotics), as well as for relief of symptoms of non-ulcer dyspepsia would be full inhibition of acid secretion. With the currently used PPI type drugs this is achieved only by intravenous infusion; in case of the drug OMEPRAZOLE this requires intravenous infusion of 8 mg per hour. Clearly, there is a need in the art for a drug or drugs acting through the mechanism of PPI-type drugs which can attain or approach full inhibition of acid secretion through oral therapy.

Because of the less than full inhibition of acid secretion and less than 24 hour inhibition through oral administration that is attained by the current dosage forms of currently used PPI-type drugs, therapy for healing of gastric and duodenal ulcerations is 4 to 8 weeks. This is in spite of the fact that the generation time of surface cells of the esophagus, stomach and duodenum is approximately 72 hours. Undoubtedly the presently observed prolonged healing times with these drugs is due to inadequate acid suppression and acid related damage. The foregoing underscores the need in the art for a drug or drugs acting through the mechanism of PPI-type drugs that can attain or approach full inhibition of acid secretion through oral therapy.

As further pertinent background to the present invention, applicants note that the concept of prodrugs that is well known in the art. Generally speaking, prodrugs are derivatives of per se drugs, which after administration undergo conversion to the physiologically active species. The conversion may be spontaneous, such as hydrolysis in the physiological environment, or may be enzyme catalyzed. From among the voluminous scientific literature devoted to prodrugs in general, the foregoing examples are cited: Design of Prodrugs (Bundgaard H. ed.) 1985 Elsevier Science Publishers B. V. (Biomedical Division), Chapter 1; Design of Prodrugs: Bioreversible derivatives for various functional groups and chemical entities (Hans Bundgaard); Bundgaard et al. Int. J. of Pharmaceutics 22 (1984) 45-56 (Elsevier); Bundgaard et al. Int. J. of Pharmaceutics 29 (1986) 19-28 (Elsevier); Bundgaard et al. J. Med. Chem. 32 (1989) 2503-2507 Chem. Abstracts 93, 137935y (Bundgaard et al.); Chem. Abstracts 95, 138493f (Bundgaard et al); Chem. Abstracts 95, 138592n (Bundgaard et al.); Chem. Abstracts 110, 57664p (Alminger et al.); Chem. Abstracts 115, 64029s (Buur et al.); Chem. Abstracts 115, 189582y (Hansen et al.); Chem. Abstracts 117, 14347q (Bundgaard et al.); Chem. Abstracts 117, 55790×(Jensen et al.); and Chem. Abstracts 123, 17593b (Thomsen et al.).

As far as the present inventors are aware, there are no prodrugs of the proton pump inhibitors presently in use. However, several United States patents describe compounds that can act as prodrugs of certain proton pump inhibitors. Specifically, U.S. Pat. No. 4,686,230 (Rainer et al.) describes derivatives of pyridylmethyl sulfinyl benzimidazoles which include a group designated “R₅” on one of the benzimidazole nitrogens. The “R₅” group is expected to cleave under physiological condition, or under the influence of an enzyme to provide the corresponding compound with a free N—H bond (see column 3 of U.S. Pat. No. 4,686,230). U.S. Pat. Nos. 5,021,433 (Alminger et al.), 4,045,563 (Berntsson et al.), 4,965,269 and Brändström et al. also describe pyridyl methyl sulfinyl benzimidazoles where one of the nitrogens of the benzimidazole moiety bears a substituent that cleaves under physiological or enzymatic conditions. U.S. Pat. No. 4,045,563 (Berntsson et al.) describes N-alkoxycarbonyl benzimidazole derivates.

A publication by Sih., et al. Journal of Medicinal Chemistry, 1991, vol. 34, pp 1049-1062, describes N-acyloxyalkyl, N-alkoxycarbonyl, N-(aminoethyl), and N-alkoxyalkyl derivatives of benzimidazole sulfoxide as prodrugs of proton-pump inhibitors. According to this article these prodrugs exhibited improved chemical stability in the solid state and in aqueous solutions, but had similar activity or less activity than the corresponding parent compounds having a free imidazole N—H group. This publication does not provide data regarding the duration of the inhibitory activity of these prodrugs.

U.S. Pat. No. 6,093,734 and PCT Publication WO 00109498 (published on Feb. 24, 2000) describe prodrugs of proton pump inhibitors which include a substituted arylsulfonyl moiety attached to one of the benzimidazole nitrogens of proton pump inhibitors having the structure identical with or related to proton pump inhibitor drugs known by the names LANSOPRAZOLE, OMEPRAZOLE, PANTOPRAZOLE and RABEPRAZOLE.

PCT Publication WO 02/30920 describes benzimidazole compounds which are said to have gastric acid secretion inhibitory and anti H. pylori effects. PCT Publication WO 02/00166 describes compounds that are said to be nitric oxide (NO) releasing derivatives of proton pump inhibitors of the benzimidazole structure.

A still further advance in the art is described in U.S. Pat. No. 6,897,227 (and in the corresponding PCT Publication No. 2004/009583 and in the corresponding European Patent No. EP 1556371). This disclosure provides prodrugs of the proton pump inhibitor type drugs of improved solubility in physiological fluids and improved cell penetration, therefore having improved efficacy in therapy of acid related diseases due to prolongation of the presence of the proton pump inhibitors in the body.

U.S. Pat. No. 5,859,030 and the publication in J. Med. Chem. Vol. 34, 1991 pages 533-541 are of further interest as background to the present invention.

The present invention provides prodrugs of the known proton pump inhibitor TENATOPRAZOLE which, unlike the other above-described proton pump inhibitors, has a methoxy substituted 1H-imidazo[4,5-b]pyridine moiety instead of the benzimidazole ring of the other proton pump inhibitors having the same type of physiological mode of action.

### SUMMARY OF THE INVENTION

The present invention relates to compounds of Formula 1 and to its positional isomer of Formula 2

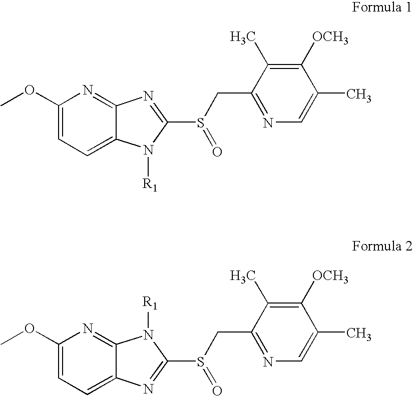

where R₁ is represented by Formula 3

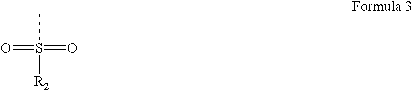

where the dashed line represents the bond connecting the sulfur atom of the formula to the nitrogen atom in the 1H-imidazo[4,5-b]pyridine nucleus. In Formula 3 the variable R₂ is phenyl, naphthyl or heteroaryl having 1 to 3 heteroatoms independently selected from N, O and S, said phenyl, naphthyl or heteroaryl groups being unsubstituted or substituted with 1 to 5 R₃ groups. R₃ is alkyl of 1 to 10 carbons, halogen substituted alkyl of 1 to 10 carbons, alkoxy having 1 to 10 carbons, halogen substituted alkoxy of 1 to 10 carbons, alkylthio having 1 to 10 carbons, halogen substituted alkylthio of 1 to 10 carbons, alkoxy carbonyl having 1 to 10 carbons, halogen substituted alkoxy carbonyl having 1 to 10 carbons, F, Cl, Br, I, NO₂, CN, OCOalkyl, NH₂, alkylamino and dialkylamino where in said OCOalkyl, alkylamino and dialkylamino groups each of said alkyl group has 1 to 10 carbons.

Alternatively and preferably the present invention relates to compounds of Formula 4 and to its positional isomer of Formula 5

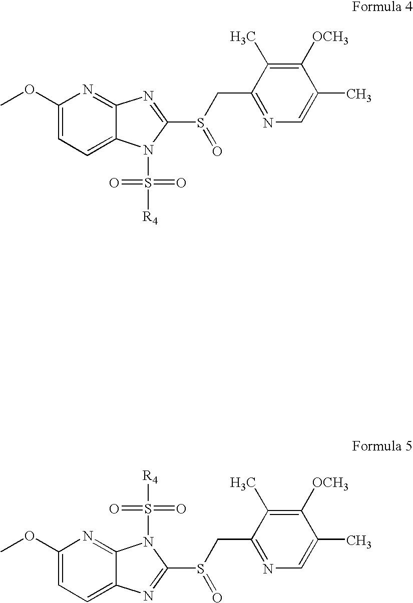

where R₄ represents the groups selected from Formulas (i) through (viii).

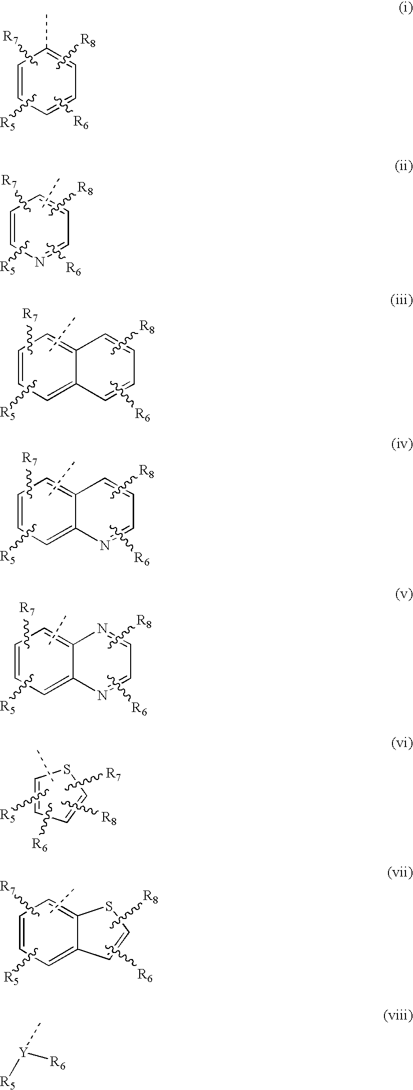

In Formulas (i) through (viii) the dashed line represents the bond connecting the R₄ group with the SO₂ group;
Y is a straight chained or branch-chained disubstituted alkyl group of 1 to 8 carbons, or Y is N;
R₅ and R₆ independently are H, a straight chained or branch-chained di- or trisubstituted alkyl group of 1 to 12 carbons including 1 or two R₉ groups, or a straight chained or branch-chained saturated hydrocarbon skeleton having no more than 12 carbons including 1 or two R₉ groups and optionally further including one to three X groups where X is independently selected from the group consisting of —O—, —S—, —NR₁₀—, —NHCO—, —CONH—, —CONHCO—, —COO—, —OCO— and a disubstituted phenyl group which can optionally be substituted with one or two halogen atoms or with one or two R₇ groups; or the R₉ group is directly attached without an intervening R₅ or R₆ group to the aromatic or heteroaromatic ring or to the V group of formulas (i) through (viii);
R₇ and R₈ independently are H, alkyl of 1 to 3 carbons, fluoroalkyl of 1 to 3 carbons, O-alkyl of 1 to 3 carbons, O-fluoroalkyl of 1 to 3 carbons, S-alkyl of 1 to 3 carbons, S-fluoroalkyl of 1 to 3 carbons;
R₉ is independently H, COOH or a tetrazole moiety;
R₁₀ is H or alkyl of 1 to 3 carbons;
with the provisos that
at least one the R₅ and R₆ groups is not H, and
at least one R₉ is not H and no more than two R₉ groups are COOH or tetrazole whereby the compound includes at least one but no more than two COOH or tetrazole groups;
when Y is —N then neither of the R₅ and R₆ groups is H,
or a pharmaceutically acceptable salt of said compound.

### DETAILED DESCRIPTION OF THE INVENTION

### Definitions

The term alkyl refers to and covers any and all groups which are known as normal alkyl, branched-chain alkyl, cycloalkyl and also cycloalkyl-alkyl.

A pharmaceutically acceptable salt may be prepared for any compound in this invention having a functionality capable of forming a salt, such as the carboxylic acid, tetrazole or a basic (for example an amine) functionality of the compounds of the present invention. A pharmaceutically acceptable salt is any salt that retains the activity of the parent compound and does not impart any deleterious or untoward effect on the subject to which it is administered and in the context in which it is administered.

Pharmaceutically acceptable salts may be derived from organic or inorganic bases. The salt may be a mono or polyvalent ion. Of particular interest are the inorganic ions, lithium, sodium, potassium, calcium, and magnesium. Organic salts may be made with amines, particularly ammonium salts such as mono-, di- and trialkyl amines or ethanol amines. Salts may also be formed with caffeine, tromethamine and similar molecules. Hydrochloric acid or some other pharmaceutically acceptable acid may form a salt with a compound of the invention when the compound includes a basic group, such as an amine or a pyridine ring.

Some of the compounds of the present invention may contain one or more chiral centers and therefore may exist in enantiomeric and diastereomeric forms. The scope of the present invention is intended to cover all isomers per se, mixtures of diastereomers and racemic mixtures of enantiomers (optical isomers) as well.

### General Embodiments

The chemical structure of the compounds of the invention is shown and described in broad terms in the Summary of the Invention in connection with Formulas 1 through 5. As it can be seen in these formulas, the compounds of the invention are pyridyl methyl sulfinyl 1H-imidazo[4,5-b]pyridines substituted in the pyridine and in the 1H-imidazo[4,5-b]pyridine moieties in the same manner as in the known proton pump inhibitor drug TENATOPRAZOLE (U.S. Pat. No. 4,808,596). The specifications of U.S. Pat. No. 4,808,596 is expressly incorporated herein by reference.

Moreover, as it can be seen in connection with Formulas 1, 2, 4 and 5, in the compounds of the invention one of the 1H-imidazo[4,5-b]pyridine nitrogens is substituted with a group, designated R₁ (═R₂SO₂) in Formulas 1 and 2 and R₄SO₂ in Formulas 4 and 5, that gradually cleave under physiological conditions and thereby provide the pyridyl methyl sulfinyl 1H-imidazo[4,5-b]pyridine compound which has a free NH function in the 1H-imidazo[4,5-b]pyridine moiety. The compound thus obtained by cleavage of the R₂SO₂ or R₄SO₂ group, as applicable, then undergoes the acid catalyzed rearrangement and provides the thiophilic species which inhibits the H,K-ATPase enzyme involved in gastric acid production. Thus, the novel compounds of the present invention bearing the R₂SO₂ or R₄SO₂ group, as applicable, are prodrugs of the proton pump inhibitor compound which could also be depicted by Formula 1 where, however instead of the R₂SO₂ group there would be a hydrogen. For further description of prodrugs of pyridyl methyl sulfinyl benzimidazoles type proton pump inhibitor drugs which include a substituted arylsulfonyl or substituted heteroarylsulfonyl moiety attached to one of the benzimidazole nitrogens reference is made to U.S. Pat. Nos. 6,093,734 the specification of which is expressly incorporated herein.

Compounds of the present invention shown in Formulas 4 and 5, in addition to having the advantage of being prodrugs of TENATOPRAZOLE also have the further advantage of having an acidic moiety (designated R₉ in these formulas. The acidic moiety (a carboxylic acid or alternatively tetrazole) provides increased solubility in physiological media and therefore is expected to make the prodrug significantly more bio-available relative to the prodrugs shown in Formulas 1 and 2 or described in U.S. Pat. No. 6,093,734. For further description of prodrugs of pyridyl methyl sulfinyl benzimidazoles type proton pump inhibitor drugs which include a carboxylic acid (or tetrazole) bearing arylsulfonyl or heteroarylsulfonyl moiety attached to one of the benzimidazole nitrogens reference is made to U.S. Pat. No. 6,897,227 the specification of which is expressly incorporated herein.

Referring now to the group designated R₂SO₂ in connection with Formulas 1 and 2 it will be apparent to those skilled in the art that this group represents the principal novel structural feature of the present invention. Among the R₂ groups phenyl is preferred, substituted or unsubstituted with the R₃ group. When the phenyl group (R₂) is substituted, then the substituent (R₃) is preferably selected from Cl, Br, F, lower alkyl, lower alkoxy, trifluoromethyl, trifluoromethoxy, di-(lower alkyl)amino, and lower alkoxycarbonyl. Even more preferably the phenyl group is unsubstituted (R₃ is H) or the substituent of the phenyl (R₂) group is selected from Cl, Br, F, methyl, methoxy, trifluoromethyl, trifluoromethoxy, dimethylamino and ethoxycarbonyl groups. Preferably there is only one R₃ substituent (other than hydrogen) in the phenyl (R₂) moiety, and preferably the R₃ substituent is in a position para (1,4) or meta (1,3) to the sulfonyl (SO₂) group.

The most preferred compounds in accordance with Formulas 1 and 2 are those wherein the R₂ phenyl group is mono-substituted either in the 4 (para) or in the 3 (meta) position with a Cl, Br, F, CH₃, CH₃O, CF₃, CF₃O—, (CH₃)₂N or EtOCO group.

The compounds of the invention in accordance with Formulas 1 and 2 can be prepared by the reacting the compound known as TENATOPRAZOLE with an arylsulfonyl chloride or heteroarylsulfonyl chloride. As noted above the known compound TENATOPRAZOLE is described in U.S. Pat. No. 4,808,596 (incorporated herein by reference) and has the structure corresponding to Formula 1 wherein the R₁ group would be H. In a broad sense the arylsulfonyl chloride or heteroarylsulfonyl chloride reagent is described by the formula R₂SO₂Cl where the R₂ group is defined as in connection with Formula 1. Reaction Scheme 1 discloses a process for preparing compounds of the invention of Formula 1 and 2 by reacting the arylsulfonyl chloride or heteroarylsulfonyl chloride reagent R₂SO₂Cl with the compound known as TENATOPRAZOLE. The reaction is typically conducted in an inert organic solvent, such as dichloromethane in the presence of an organic base, such as triethylamine. As it can be seen in Reaction Scheme 1, the aryl or heteroaryl sulfonylation reaction gives rise to the two isomeric or products shown by Formulas 1a and 1b, respectively. Depending on the specific structure and reaction conditions the positional isomers may be formed in substantially equal or in other varying ratios.

The arylsulfonyl chloride or heteroarylsulfonyl chloride reagent R₂SO₂C1 can be obtained in accordance with procedures well known in the art.

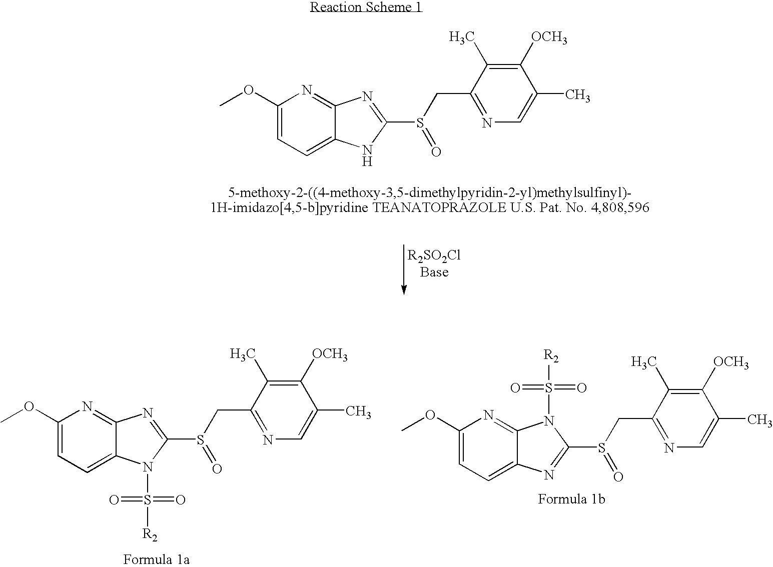

In the further description below, a synthetic process is shown whereby a single isomer corresponding to Formula 1 can be obtained. For further description of the synthetic methods for obtaining the compounds of the invention in accordance with Formulas 1 and 2 and for the preferred substitutions in their arylsulfonyl or heteroarylsulfonyl moiety reference is made to U.S. Pat. No. 6,093,734, expressly incorporated herein by reference.

Referring now to the compounds of the invention shown by Formulas 4 and 5, as noted above, these compounds have the additional preferred feature of including at least one acidic function which renders the compounds, or their salts, more soluble in physiological fluids and improves their bioavailability. Among these compounds the preferred ones are where the group designated R₄ shown by structural formulas (i) through (viii), represents a substituted phenyl (formula (i)), substituted pyridyl (formula (ii)), substituted naphthyl (formula (iii)) or substituted thienyl (formula (vi)). Presently still more preferred are compounds where R₄ represents substituted phenyl (formula (i)).

Referring now to the variables designated R₇ and R₈ in formulas (i) through (vii), compounds of the invention are presently preferred where these variables are independently selected from H, methyl, ethyl, iso-propyl, methoxy, ethoxy, CF₃, CH₃O and OCF₃. Preferably these substituents are on the carbon or carbons which is or are located adjacent to the carbon linked to the sulfonyl group. In many of the presently preferred compounds of the invention where R₄ is phenyl, these carbons are in ortho position relative to the sulfonyl group. As it will be recognized by those skilled in the art, the R₇ and R₈ substituents can electronically and sterically influence the rate of cleavage or hydrolysis of the sulfonyl group from the 1H-imidazo[4,5-b]pyridine nucleus, and thereby influence the bio-availability of the prodrugs of the present invention. When, as in several preferred embodiments, the R₄ and R₈ substituents are in ortho (or comparable) position relative to the sulfonyl group, then the steric bulk or lack of steric bulk of these substituents are especially significant in influencing the rate of hydrolysis of the sulfonyl group from the 1H-imidazo[4,5-b]pyridine moiety. In several of the presently preferred compounds of the invention R₄ is phenyl (formula (I)) and R₇ and R₈ are both methyl and occupy the ortho and ortho′ positions on the phenyl ring. In other examples of the preferred compounds of the invention R₄ is phenyl, one of the R₇ and R₈ groups is H, and the other is iso-propyl. The ortho and ortho′ methyl and the ortho iso-propyl substituents slow down the rate of hydrolysis relative to a compound of otherwise comparable structure that lacks these ortho and ortho′ substituents.

Referring now to the variables R₅ and R₆ it is an important feature of the compounds of Formulas 4 and 5 of the present invention that one or both of these groups include a carboxylic acid (or like-wise acidic tetrazole) function. The purpose of function of the carboxylic acid moiety included in these variables in the compounds of the invention is to render the compounds more soluble in aqueous physiological fluids at physiological pH than the prodrugs of pyridyl-methyl-sulfinyl-benzimidazole proton pump inhibitors of several prior art disclosures. Generally speaking it is desired within the scope of the present invention that the pKa of the carboxylic acid (or tetrazole) moiety of the compounds of the invention be in the range of 2 to 6, even more preferable the pKa should be in the range of 2 to 4, and still more preferably the pKa is approximately 3. One or two carboxylic acid moieties attached to the R₅ and/or to the R₆ substituents provide the desired pKa and therefore the desired solubility to the compounds of the invention. Those skilled in the art will recognize that the acidity of the carboxylic acid moiety is influenced by the electronic effects of other groups in its vicinity, particularly so when the carboxylic acid moiety is attached directly to an aromatic ring. It will also be recognized that tetrazole ring may substitute for one or both carboxylic acid moieties, and further that physiologically acceptable salts of the compounds of the present invention may have the same or even better solubility in physiological fluids than the corresponding free acids.

The requirement of the compounds of Formula 4 and of Formula 5 of the present invention that one or both of the R₅ and R₆ groups include at least one but no more than a total of two carboxylic acid (or tetrazole) moieties (or its pharmaceutically acceptable salt) can be satisfied with a large variety or combination of R₅ and R₆ groups. For example, one of these two groups may represent H, in which case the other group includes one or two carboxylic acid (or tetrazole) function (or its pharmaceutically acceptable salt). Alternatively, each of the R₅ and R₆ may include one carboxylic acid (or tetrazole) function (or their pharmaceutically acceptable salt). The carboxylic acid (or tetrazole) functions, designated R₉ in connection with formulas (i) through (viii), may be directly attached to the aromatic or heteroaromatic rings (formulas (i) through (vii)) or to the Y group (formula (viii)), or one or both of the R₅ and R₆ groups may include a hydrocarbon “skeleton” or “frame” which is attached directly to the aromatic rings (formulas (i) through (vii)), or to the Y group (formula (viii)). Alternatively, the hydrocarbon “skeleton” or “frame” itself may be attached to the aromatic or heteroaromatic rings (formulas (i) through (vii)) or to the Y group (formula (viii)) through an intermediate ether, thioether, amino, ester or amide function. These functions are represented by the variable X in the description of the compounds in the Summary Section of this application for patent.

Moreover, the ether, thioether, amino, ester or amide function or functions may be included at one or more places in the hydrocarbon “skeleton” or “frame” in which case the carboxylic acid (or tetrazole) moiety or moieties are attached to or are “carried by” R₅ and/or R₆ groups which themselves include ether, thioether, amino, ester or amide linkages. Any combination of these linkages may be suitable for providing compounds within the scope of the invention. Moreover, the “skeleton” or “frame” itself may be straight chained or branch chained, and branching may be due to carbon-to-carbon or to carbon-to-X group linkages.

With the understanding that the R₅ and R₆ groups may be independently selected and in such a manner that at least one but no more than two carboxylic acid (or tetrazole) function is present in the compounds of the invention, the following serve as examples for preferred embodiments of the R₅ and R₆ groups:

### (1) H,

(2) (CH₂)ₘR₉,
(3) (CH₂)ₘCH(R₉)(CH₂)ₙ[CH(R₉)]_{q}(CH₂)ₒCH₃,
(4) X(CH₂)ₘR₉,
(5) X(CH₂)ₘCH(R₉)[(CH₂)ₙCH(R₉)]_{q}(CH₂)ₒCH₃,
(6) (CH₂)ₘX(CH₂)ₚCH(R₉)[(CH₂)ₙCH(R₉)]_{q}(CH₂)ₒCH₃,
(7) (CH₂)ₘCH(R₉)(CH₂)ₙX[(CH₂)ₚCH(R₉)]_{q}(CH₂)ₒCH₃,
(8) (CH₂)ₘX[(CH₂)ₚCH(R₉)]_{q}(CH₂)ₙCH(R₉)(CH₂)ₒCH₃,
(9) (CH₂)ₘ[CH(R₉)(CH₂)ₙ]_{q}X(CH₂)ₚCH(R₉)(CH₂)ₒCH₃,
(10) X(CH₂)ₘXCH(R₉)[(CH₂)ₙCH(R₉)]_{q}(CH₂)ₒCH₃,
(11) (CH₂)ₘ[CH(CH₂)ₛ(R₉)](CH₂)ₙ[CH(CH₂)ₛ(R₉)]_{q}(CH₂)ₒCH₃,
(12) X[CH(CH₂)ₛ(R₉)]ₘR₉,
(13) X(CH₂)ₘ[CH(CH₂)ₛ(R₉)][CH(CH₂)ₛ(R₉)]_{q}(CH₂)ₒCH₃,
(14) (CH₂)ₘX(CH₂)ₚ[CH(CH₂)ₛ(R₉)][CH(CH₂)ₛ(R₉)]_{q}(CH₂)ₒCH₃,
(15) (CH₂)ₘ[CH(CH₂)ₛ(R₉)](CH₂)ₙX[CH(CH₂)ₛ(R₉)]_{q}(CH₂)ₒCH₃,
(16) (CH₂)ₘX[(CH₂)ₚ[CH(CH₂)ₛ(R₉)]]_{q}(CH₂)ₙ[CH(CH₂)ₛ(R₉)](CH₂)ₒCH₃,
(17) (CH₂)ₘ[[CH(CH₂)ₛ(R₉)](CH₂)ₙ]_{q}X(CH₂)_{P}[CH(CH₂)ₛ(R₉)](CH₂)ₒCH₃,
(18) X(CH₂)ₘX[CH(CH₂)ₛ(R₉)][(CH₂)ₙ[CH(CH₂)ₛ(R₉)]]_{q}(CH₂)ₒCH₃
(19) X(CH₂)ₘC(CH₃)₂R₉
(20) X(CH₂)ₘX(CH₂)ₙR₉
where m is an integer having the values 0 to 6;
n is an integer having the values 0 to 5;
q is an integer having the values 0 or 1, and
o is an integer having the values 0 to 5,
s is an integer having the values 0 to 5,
the sum of the integers m, n, q, o and s does not exceed 12, and
where the other variables have the meaning defined above in connection with Formulas 4 and 5,
the provisos set forth in connection with Formulas 4 and 5 apply with the further proviso that the R₉ group is not directly linked to O, S, NR₁₀, NHCO, CONH, COO or OCO group.

Utilizing the substituted phenylsulfonyl group and COOH (for R₉) as preferred moieties in the compounds of the present invention, the structural formulas which show, not as a limitation but for illustration and exemplary purposes, the preferred R₅ or R₆ groups designated (2), (3), (4), (5), (10), (11), (12), (19) and (20) above, are shown in Columns 9 and 10 of U.S. Pat. No. 6,897,227, expressly incorporated herein by reference. In this reference patent R₁, R₂, R₃ and R₄ correspond to the variables identified as R₅, R₆, R₇ and R₈ in this disclosure. For the most preferred groups corresponding to the R₄SO₂ group of Formulas 4 and 5 of this disclosure see also the groups shown in Columns 10 through 14 of U.S. Pat. No. 6,897,227.

A general route for the synthesis of the compounds of Formula 4 and of Formula 5 of the present invention is shown below in Reaction Scheme 2 where the starting material is the known compound TENATOPRAZOLE, and for the simplicity of illustration the aryl, heteroaryl or alkyl moieties of formulas (i) through (viii) are illustrated by a phenyl group, only the R₅ group (as defined above) and only a monocarboxylic acid are shown. However, those skilled in the art will readily understand that the herein described synthetic procedure can be applied to the preparation of all compounds within the scope of the invention with only such modifications which are readily apparent to those skilled in the art in view of the present disclosure.

Thus, in accordance with Reaction Scheme 2 TENATOPRAZOLE is reacted with a chlorosulfonyl compound of Formula 6 in the presence of base such as sodium hydride, triethylamine, di(isopropyl)methylamine or other suitable base, in an aprotic solvent such as CH₂Cl₂. The compound of Formula 6 includes a substituted or unsubstituted phenylsulfonylethyl ester of the carboxylic acid moiety that is to be included in the compounds of the invention. The variable Z represents an optional substituent of the phenyl group of the phenylsulfonylethyl moiety. Generally speaking, the chlorosulfonyl compounds within the scope of Formula 6 can be obtained by those skilled in the art in light of widely available chemical patent and scientific literature. Syntheses of several examples of these reagents of Formula 6 are also described in U.S. Pat. No. 6,897,227, incorporated herein by reference. The reaction between TENATOPRAZOLE and the chlorosulfonyl compound of Formula 6 gives rise to positional isomers of Formula 7a and Formula 7b.

Referring still to Reaction Scheme 2, the intermediates of Formula 7a and 7b are hydrolyzed in the presence of mild base, such as NaHCO₃, to provide the sodium salt of the compounds of the invention. These are the positional isomers shown by the Formulas 8a and 8b. A by-product of this reaction is a substituted or unsubstituted phenyl vinyl sulfone that is also shown in Reaction Scheme 2. The sodium salt can be readily converted to the free acid compounds of the invention which are the ones actually shown (in their simplified form) by Formulas 8a and 8b.

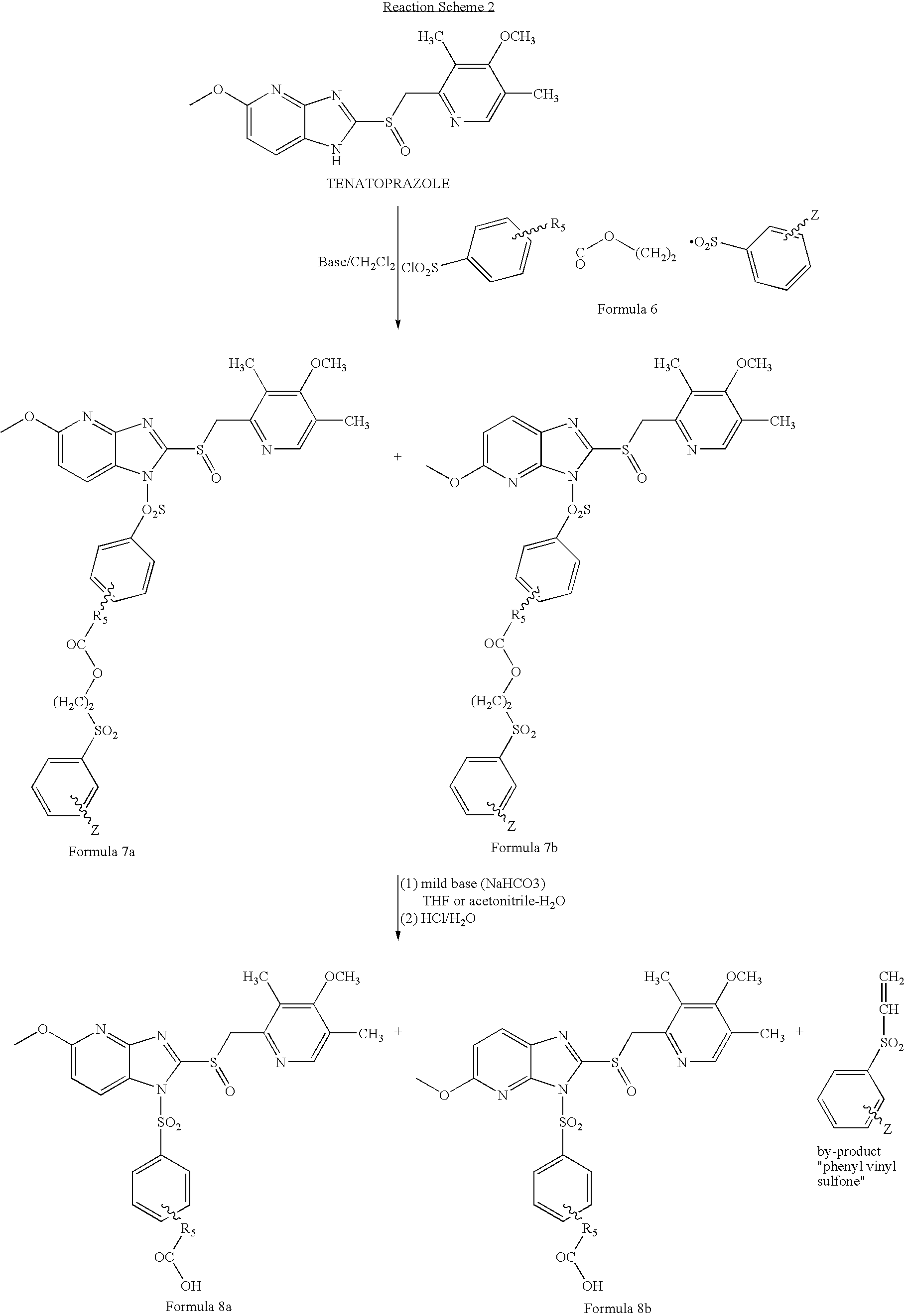

Reaction Scheme 3 illustrates a more specific example for preparing a preferred pair of isomeric compounds corresponding to Formulas 4 and 5 of the present invention. In this reaction scheme the compounds and reagents are identified by chemical names.

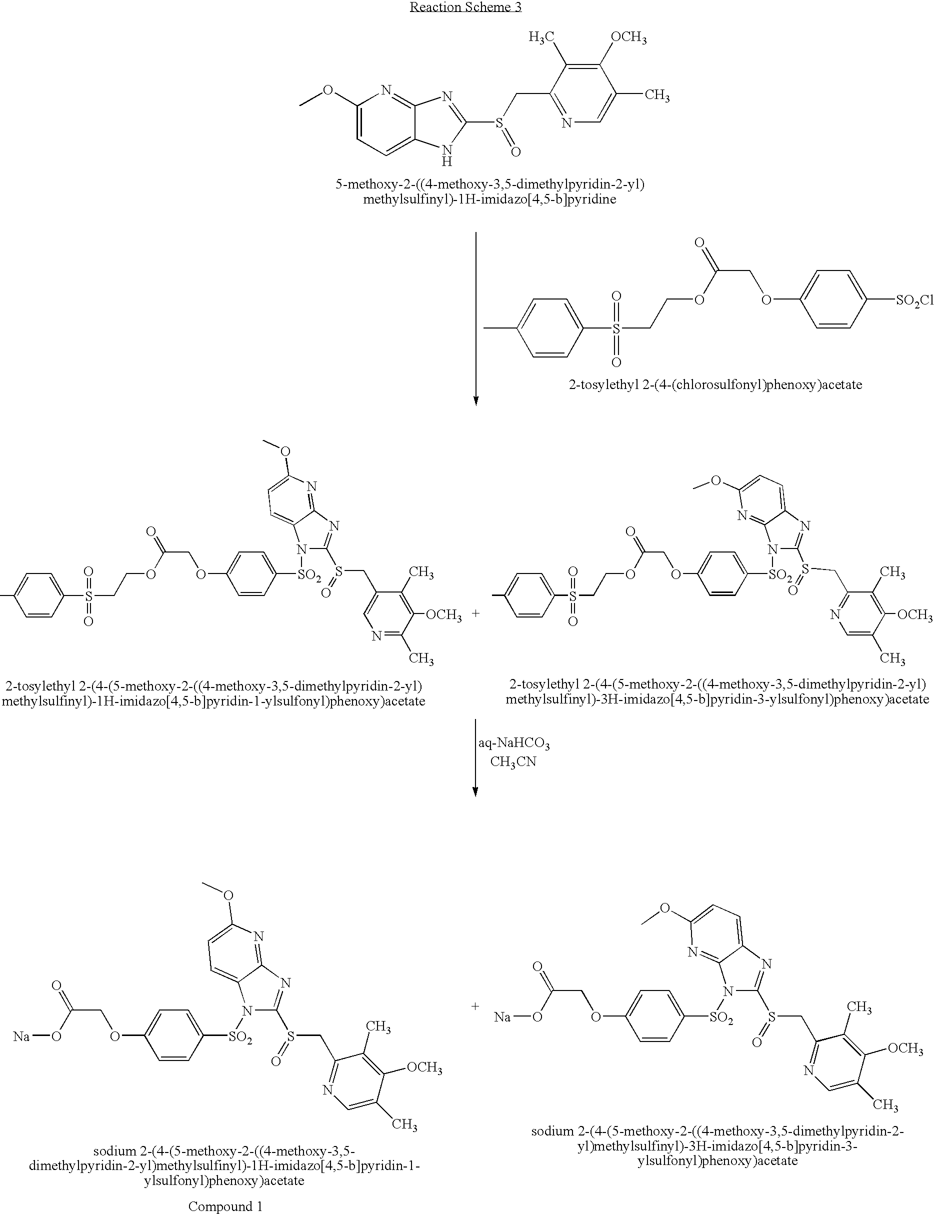

The reagent 2-tosylethyl 2-(4-(chlorosulfonyl)phenoxy)acetate can be synthesized in accordance with U.S. Pat. No. 6,897,227.

As is apparent from all of the foregoing reaction schemes, the reaction of TENATOPRAZOLE with the arylsulfonyl chloride or heteroarylsulfonyl chloride (for example R₂SO₂Cl in Reaction Scheme 1, Formula 6 in Reaction Scheme 2, 2-tosylethyl 2-(4-(chlorosulfonyl)phenoxy)acetate in Reaction Scheme 3) gives rise to 2 isomeric compounds, both of which are, generally speaking, within the scope of the invention. The two isomers are usually but not necessarily formed approximately in 1 to 1 ratios in the reaction, and it is expected in accordance with the invention that the biological activity, solubility and particularly the stability of the isomers may also differ, in some cases significantly. Although, when desired, the isomers can be separated from one another by state-of-the-art separation techniques, such as high pressure liquid chromatography (HPLC), a more efficient synthetic route to synthesize single isomers of these compounds has been developed also. Reaction Scheme 4 discloses a synthetic route to stereospecifically obtain that isomer of the prodrug of TENATOPRAZOLE which is expected to have the greater stability or the better pharmaceutical properties, or both, than the other isomer. Reaction Scheme 4 discloses the synthesis of the presently most preferred specific compound, (Compound 1). Compound 1 is within the scope of general Formula 4 and includes a carboxylic acid moiety attached to the benzenesulfonyl ring that is linked to the nitrogen in the one (1) position of the 1H-imidazo[4,5-b]pyridine ring. The numbering of the 1H-imidazo[4,5-b]pyridine ring is shown in the reaction scheme. Those skilled in the art will readily understand that the herein described stereospecific synthesis of Compound 1 can be applied to the stereospecific syntheses of all compounds within the scope of Formulas 1 and 4 with only such modification of reagents and conditions which will be readily apparent to those skilled in the art in view of the present disclosure. and in view of the disclosures of the stereospecific syntheses of other prodrugs of benzimidazole proton pump inhibitors described in U.S. Pat. No. 6,897,227, incorporated herein by reference. (See the reaction scheme in Columns 17-21, and the description of certain specific compounds in the 6,897,227 patent.)

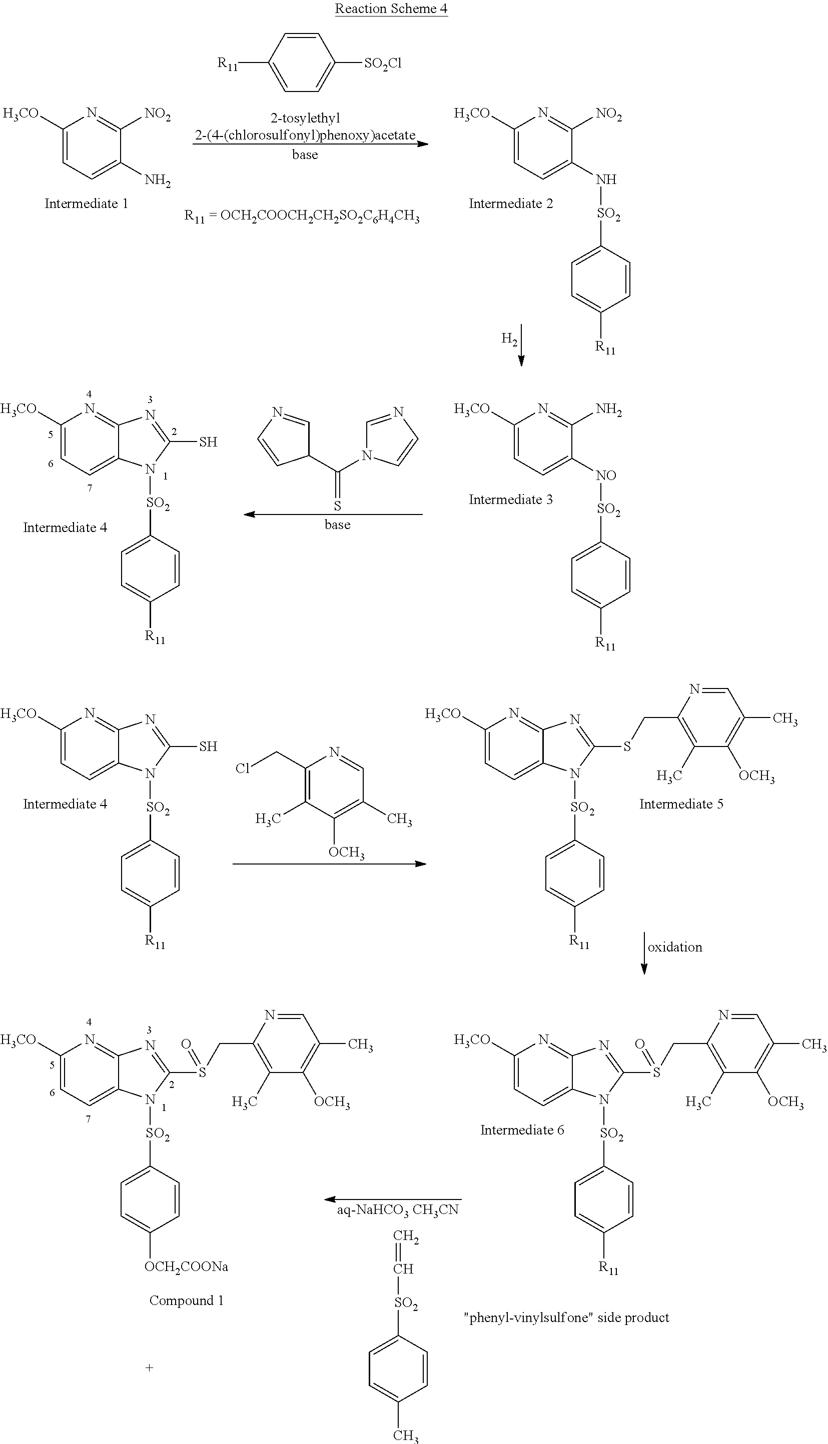

Referring now to Reaction Scheme 4,2-nitro-3-amino-6-methoxypyridine (Intermediate 1) is reacted with 2-tosylethyl 2-(4-(chlorosulfonyl)phenoxy)acetate in the presence of base to provide the benzenesulfonylated compound (Intermediate 2). Intermediate 1 can be obtained in accordance with such modifications of well known synthetic processes which will become readily apparent to those skilled in the art. The nitro group of Intermediate 2 is then reduced to provide Intermediate 3. Intermediate 3 is ring-closed by treatment with thiocarbonyldiimidazole (Im₂C═S) (or by treatment with phenylisocyanate, or with thiophosgene) to provide a 2-thio-1H-imidazo[4,5-b]pyridine derivative (Intermediate 4) where the chlorosulfonyl group is attached to the nitrogen in the 1-position. Intermediate 4 is reacted with 2-chloromethyl-4-methoxy-3,5-dimethylpyridine to give rise to a Intermediate 5. The reagent 2-chloromethyl-4-methoxy-3,5-dimethylpyridine or its hydrochloride salt can be obtained by treatment of 4-methoxy-3,5-dimethylpyridine-methanol (obtainable from Aldrich) with thionyl chloride. The thioether linkage of Intermediate 5 is oxidized to the sulfoxide level by treatment with 3-chloroperoxybenzoic acid (meta-chloroperbenzoic acid, m-CPBA) or with other suitable oxidizing agent to yield Intermediate 6. Treatment of Intermediate 6 with base saponifies the carboxylic acid ester function attached to the aryl ring of the chlorosulfonyl moiety and gives rise to the TENATOPRAZOLE derivative prodrug compound of the invention (Compound 1) plus a “phenyl-vinylsulfone” side product also shown in the reaction scheme.

### Biological Activity, Modes of Administration

A significant advantage of the compounds of the present invention is that they can release the active forms of the proton pump inhibitor spontaneously by hydrolysis in the mammalian (including human) body. Hydrolysis can occur chemically or enzymatically. Because the compounds of this invention spontaneously release the active form of the proton pump inhibitor drug by in vivo hydrolysis, they can attain longer duration of effective drug concentration in the body. Thus, the compounds of the present invention are prodrugs which are converted to the active drug by hydrolysis in the body, providing long duration of effective concentration. The long duration of inhibitory activity by spontaneous hydrolysis of the compounds of this invention allows more effective inhibition of gastric acid secretion, which enables better therapy of acid related disease defined above. Compounds of this invention can be administered for inhibiting gastric acid secretion orally. The typical daily dose of the compounds will depend on various factors such as the individual requirement of each patient. In general, oral and parenteral dosages will be in the range of 5 to 300 mg per day.

Those skilled in the art will readily understand that for oral administration the compounds of the invention are admixed with pharmaceutically acceptable excipients which per se are well known in the art. Specifically, a drug to be administered systemically, it may be confected as a powder, pill, tablet or the like or as a syrup or elixir suitable for oral administration. Description of the substances normally used to prepare tablets, powders, pills, syrups and elixirs can be found in several books and treatise well known in the art, for example in Remington's Pharmaceutical Science, Edition 17, Mack Publishing Company, Easton, Pa.

Compounds of the present invention can be combined with certain amounts of known proton pump inhibitors, e.g. LANSOPRAZOLE (U.S. Pat. No. 4,628,098), OMEPRAZOLE (U.S. Pat. Nos. 4,255,431 and 5,693,818), PANTOPRAZOLE (U.S. Pat. No. 4,758,579), RABEPRAZOLE (U.S. Pat. No. 5,045,552) ILAPRAZOLE (U.S. Pat. No. 5,554,631) or TENATOPRAZOLE (U.S. Pat. No. 4,808,596) to provide a drug-prodrug combination, and the combination can be administered for inhibition of gastric acid secretion. Thus, initially the proton pump inhibitor (drug) inhibits gastric acid secretion of the patient. The aforesaid known and widely used proton pump inhibitors have 60-90 minutes of plasma half-life. As the effective concentration of the proton pump inhibitor (drug) is decreased by metabolism, the compound of the present invention (prodrug) continuously undergoes hydrolysis and provides and maintains new active inhibitor concentration in the mammalian, including human body for much longer periods of time than the unmodified proton pump inhibitor. This results in more rapid and effective inhibition of acid secretion.

A disadvantage of many of the presently used proton pump inhibitors is that for therapy by injection in a liquid form they must be reconstituted from a lyophilized powder in a medium having the high pH of approximately 9.5 to 10.5. The prodrugs of the present invention having the chemical structure of Formulas 4 and 5 also overcome the disadvantage of requiring a reconstituting medium having such high pH, because these preferred compounds of the present invention can be reconstituted to form an injectable liquid in a medium of approximately pH 7 to 8. It will be readily appreciated by those skilled in the art that for administration in liquid form by injection the liquid that reconstitutes the drug is a pharmaceutically acceptable aqueous solution that per se is known in the art. Such pharmaceutically acceptable solutions utilized for administration of drugs in injectable form are described for example in the treatise PHARMACEUTICAL DOSAGE FORMS (Parenteral Medications, Volume 1, Edited by K. E. Avis, H. A. Lieberman and L. Lachman (1992).

Among the benefits of the pre-proton pump inhibitor (P-PPI) type of drugs of the present invention is their ability to provide more effective treatment of erosive esophagitis and of less severe reflux diseases as well. This is because effective treatment of erosive esophagitis (and to a lesser extent of lesser reflux diseases) requires prevention of the reflux of gastric contents at pH 3.0 or still lower pH. The current PPI drugs allow several acidic excursions to pH<2.0 per day, resulting in often a moderate to weak amelioration of symptoms. However, healing would require elevation to pH>4.0 for about 16 hours per day or longer. When, as in current usual treatment by PPIs, the other 8 hours contain episodic acidity to pH 3.0 or less, the patients tend to continue to complain of pain. The more effective and more continues acid suppression by the drugs of the present invention is likely to result in substantially better treatment of this disease, as well as faster healing of all acid related erosions or ulcers.

The pre-proton pump inhibitor (P-PPI) type of drugs of the present invention provides improved dual therapy for H. pylori eradication. This is because the PPI's synergize with cell division dependent antibiotics such as amoxicillin (cell wall biosynthesis) and clarithromycin (protein synthesis) by elevating gastric surface pH to enable a larger fraction of the bacterial population to be in dividing or growth phase during presentation of the antibiotic to the gastric lumen. However, their effect on intragastric pH is limited by their dwell time in the plasma. The pre-proton pump inhibitor (P-PPI) type of drugs of the present invention can continuously elevate intra gastric pH close to neutrality on current once a day therapy. Therefore, 100% eradication of the bacteria is expected in dual therapy with the prodrugs of the invention (a prodrug of TENATOPRAZOLE in accordance with the invention) plus an effective antibiotic, such as amoxicillin.

Even monotherapy for H. pylori eradication is likely to be successful with the pre-proton pump inhibitor (P-PPI) type of drugs of the present invention. This is because in the absence of acid, the enzyme H. pylori urease elevates environmental pH to >8.3, which is toxic to the organism. PPI's in current formulation inhibit growth or present of the organism in the antrum, due to elevation of antral pH to close to neutrality. Elevation of 24 hour pH to neutrality, as it can be accomplished with the drugs of the present invention, is likely to result in “self eradication” of the bacteria.

Approximately 30% of patients with gastrointestinal distress appear with symptoms without quantitative underlying disease (non-ulcer dyspepsia). The most likely cause for these symptoms is upper gastrointestinal afferent nerve sensitivity to gastric acid. Only highly effective inhibition of acid secretion or even acid ablation can ameliorate these symptoms and this can be attained with the drugs of the present invention.

### Solubility and Stability

### Solubility

A further significant advantage of the proton pump inhibitor prodrugs of the present invention which have the chemical structure shown in Formulas 4 and 5 (preferred compounds) relative to the proton pump inhibitor prodrugs disclosed in U.S. Pat. No. 6,093,734, 6,559,167 and PCT Publication WO 00109498 is their increased solubility. To illustrate this, the aqueous solubility of each of the prior art compounds (a) through (f) shown below is less than 0.01 μg per milliliter (<0.01 μg/mL) when these prior art compounds are prodrugs of the drug LANSOPRAZOLE (compounds (a) through (c), and between 5 to 8 μg per milliliter (5 to 8 μg/mL) when these prior art compounds are prodrugs of the drug OMEPRAZOLE (compounds (d) through (f). In contrast, the solubility in distilled water of the sodium salt of the carboxylic acid of Compound 1 of the invention in pure isomeric form or mixed with its positional isomer, is expected to be significantly greater than of these prior art prodrugs, probably reaching or exceeding 100 μg per milliliter (>100 μg/mL).

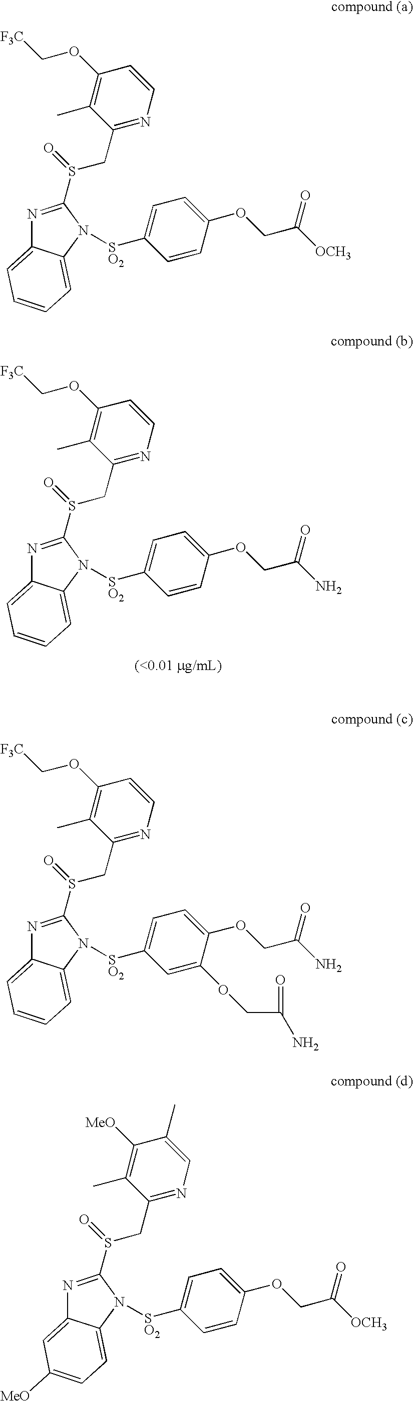

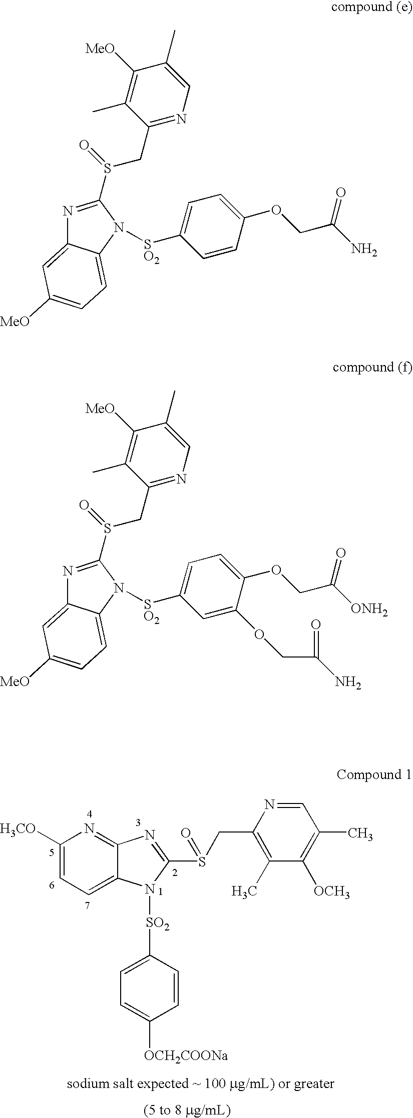

The solubility of the sodium salts of certain exemplary compounds of the present invention which have the chemical structure shown in Formulas 4 and 5 in a phosphate buffered saline buffer of pH 7.4 (50 mM sodium phosphate, 10 mM KCl, 0.1 M NaCl) and also in a more acidic Britton Robinson buffer of pH 3 (40 mM acetic acid, 40 mM phosphoric acid, 40 mM boric acid, 36 mM NaOH, and 19.6 mg/ml KCl) can also be evaluated. These compounds are expected to be highly soluble in the phosphate buffered saline buffer at pH 7.4. These compounds are expected to be less soluble little in the Britton Robinson buffer of pH 3, but still more soluble than the prior art compounds (a) through (f). This is understandable in view of the fact that in the buffer of pH 7.4 the compounds of the invention form a sodium salt, whereas in the buffer of pH 3 the compounds are less soluble free carboxylic acids. Thus, a solution of a sodium or other pharmaceutically acceptable salt of these preferred compounds of the invention, or stated in an other way these preferred compounds of the invention at pH 7 or above are expected to be highly soluble and therefore suitable for administration by intravenous injection.

### Stability in Buffers

The stability of the compounds which have the chemical structure shown in Formulas 4 and 5 (preferred compounds) in aqueous solution (0.1 mg/mL) can be investigated in Britton Robinson buffers of pH 3, pH 7, and pH 9, respectively. A solution of 0.1 mg/mL concentration of each compound in each buffer is prepared and the solutions are stored at 37° C. for 1 h and then the concentrations of test compounds are determined by HPLC. It is expected that the preferred compounds are stable in aqueous solution under neutral conditions. Half-life of hydrolysis at pH 7 of these prodrugs to yield the corresponding proton pump inhibitory drugs is expected to be over 50 hours. Therefore these prodrugs per se are expected to be stable enough for intravenous injection.

### Stability in Plasma

Exemplary test compounds (prodrugs) of the invention can be incubated in plasma at 37° C. to test their stability and hydrolysis to give the corresponding drug TENATOPRAZOLE. The concentration of the compounds and also of the corresponding proton pump inhibitor TENATOPRAZOLE can be determined by a gradient HPLC-UV method. Although the half-lives of test compounds in plasma is expected to vary depending on their specific structure, nevertheless it is expected that in plasma the compounds of the invention are converted into the corresponding proton pump inhibitor TENATOPRAZOLE at a rate faster than the hydrolysis rate of the compound at neutral or near neutral pH. For example, a 1:1 mixture of sodium 2-(4-(5-methoxy-2-((4-methoxy-3,5-dimethylpyridin-2-yl)methylsulfinyl)-1H-imidazo[4,5-b]pyridin-1-ylsulfonyl)phenoxy)acetate (Compound 1) and sodium 2-(4-(5-methoxy-2-((4-methoxy-3,5-dimethylpyridin-2-yl)methylsulfinyl)-3H-imidazo[4,5-b]pyridin-3-ylsulfonyl)phenoxy)acetate was rapidly hydrolyzed with a half-life of 1.2 min in the rat plasma at 37° C. to provide tenatoprazole. In human plasma, half-life of (Compound 1) was 1.35 min. The half-life of a 1:1 mixture of sodium 2-(4-(5-methoxy-2-((4-methoxy-3,5-dimethylpyridin-2-yl)methylsulfinyl)-1H-imidazo[4,5-b]pyridin-1-ylsulfonyl)phenoxy)acetate and sodium 2-(4-(5-methoxy-2-((4-methoxy-3,5-dimethylpyridin-2-yl)methylsulfinyl)-3H-imidazo[4,5-b]pyridin-3-ylsulfonyl)phenoxy)acetate in physiological pH 7.4 without the plasma or blood was greater than 24 hr. Therefore the compounds of the invention and especially the preferred compounds which have the chemical structure shown in Formulas 4 and 5 are good candidates to be used as prodrugs in intravenous injection and are expected to release the active proton pump inhibitor in vivo.

### Assays For Inhibitory Effect Of The Compounds Of The Invention On Gastric Acid Secretion Of The Conscious Male Rat At Timed Intervals

### Oral Administration

Male rats (the Sprague-Dawley strain) are used in this assay. Salts of known benzimidazole type proton pump inhibitors, such as OMEPRAZOLE sodium salt, LANSOPRAZOLE sodium salt or TENATOPRAZOLE sodium salt (20 mg) is suspended in 10 ml of 0.1 N NaHCO₃. 70 mg of each of the sodium salts of the compounds of the present invention are dissolved in 6 ml of 50% DMSO-50% 50 mM sodium phosphate buffer, pH 7.4. The doses administered to the rats are as follows (μmole per kg body weight of the rat): TENATOPRAZOLE (40 μmole/kg), a mixture of sodium 2-(4-(5-methoxy-2-((4-methoxy-3,5-dimethylpyridin-2-yl)methylsulfinyl)-1H-imidazo[4,5-b]pyridin-1-ylsulfonyl)phenoxy)acetate (Compound 1) and sodium 2-(4-(5-methoxy-2-((4-methoxy-3,5-dimethylpyridin-2-yl)methylsulfinyl)-3H-imidazo[4,5-b]pyridin-3-ylsulfonyl)phenoxy)acetate (1:1 of the isomeric mixture) 20 μmole/kg, and 40 μmole/kg. These drug solutions are orally administered. 3 hours after administration the abdomen of the rat is incised and the pylorus is ligated under light ether anethesia. Histamine (40 mg/kg) and carbachol (4 μg/kg) are subcutaneously injected for acid stimulation. Immediately the abdomen is closed. Two hours later, the esophagus is ligated and the stomach is removed. The gastric juice is collected and acid output is quantified by titration using 0.1 N NaOH solution. As a control experiment, 1 ml of 50 mM phosphate buffer (pH 7.4) solution is orally administrated without any prodrug or proton pump inhibitory drug. Acid output is quantified by same method as described above, showing maximum histamine and carbachol-stimulated gastric acid secretion. Percentage inhibition is calculated from the fractional responses elicited by the test compound and a control experiment.

Inhibition of a mixture of sodium 2-(4-(5-methoxy-2-((4-methoxy-3,5-dimethylpyridin-2-yl)methylsulfinyl)-1H-imidazo[4,5-b]pyridin-1-ylsulfonyl)phenoxy)acetate (Compound 1) and sodium 2-(4-(5-methoxy-2-((4-methoxy-3,5-dimethylpyridin-2-yl)methylsulfinyl)-3H-imidazo[4,5-b]pyridin-3-ylsulfonyl)phenoxy)acetate (1:1 of the isomeric mixture) at a dose of 40 μmole/kg was 83.3±13.7%.
A mixture of sodium 2-(4-(5-methoxy-2-((4-methoxy-3,5-dimethylpyridin-2-yl)methylsulfinyl)-1H-imidazo[4,5-b]pyridin-1-ylsulfonyl)phenoxy)acetate (Compound 1) and sodium 2-(4-(5-methoxy-2-((4-methoxy-3,5-dimethylpyridin-2-yl)methylsulfinyl)-3H-imidazo[4,5-b]pyridin-3-ylsulfonyl)phenoxy)acetate (1:1 of the isomeric mixture) provided higher plasma concentration of TENATOPRAZOLE than TENATOPRAZOLE at 5 h of postdose. Male rats (the Sprague-Dawley strain) are used in this assay. Plasma concentration of tenatoprazole at 5 h of postdose of a mixture of sodium 2-(4-(5-methoxy-2-((4-methoxy-3,5-dimethylpyridin-2-yl)methylsulfinyl)-1H-imidazo[4,5-b]pyridin-1-ylsulfonyl)phenoxy)acetate (Compound 1) and sodium 2-(4-(5-methoxy-2-((4-methoxy-3,5-dimethylpyridin-2-yl)methylsulfinyl)-3H-imidazo[4,5-b]pyridin-3-ylsulfonyl)phenoxy)acetate (1:1 of the isomeric mixture) (40 μmole/kg) was 1940±1685 ng/ml. However, TENATOPRAZOLE at the same dose provided 1759±810 ng/ml at 5 h of postdose. This shows that a mixture of sodium 2-(4-(5-methoxy-2-((4-methoxy-3,5-dimethylpyridin-2-yl)methylsulfinyl)-1H-imidazo[4,5-b]pyridin-1-ylsulfonyl)phenoxy)acetate (Compound 1) and sodium 2-(4-(5-methoxy-2-((4-methoxy-3,5-dimethylpyridin-2-yl)methylsulfinyl)-3H-imidazo[4,5-b]pyridin-3-ylsulfonyl)phenoxy)acetate (1:1 of the isomeric mixture) maintains higher plasma level of TENATOPRAZOLE with longer plasma half-life than that of TENATOPRAZOLE.

### Intravenous Administration

Inhibition of gastric acid secretion after intravenous administration of compounds of the invention can be assayed as follows. TENATOPRAZOLE sodium salt (20 mg) or the sodium salt of other known proton pump inhibitor drug, such as LANSOPRAZOLE sodium salt (20 mg) is dissolved in 40% hydroxypropyl-beta-cyclodextrin. The preferred compounds of the invention used in this experiment are dissolved in phosphate buffered saline solution of pH 7.4. Each compound is injected intravenously at a dose of 5 μmole/kg or 10 μmole/kg (mole per kg body weight of rat). Between 2 to 4 hours after injection gastric juice is collected, and the percentage of inhibition is determined as described above.

### EXPERIMENTAL

The following description of synthesis and other experimental results refers to the compounds shown in Reaction Scheme 3.

To a heterogeneous solution of TANATOPRAZOLE (2.5 g, 7.22 mmol) in 20 mL of CH₂Cl₂ was added triethylamine (4 mL) at room temperature, in which time the mixture became homogeneous. To the clear reaction mixture was added the (p-chlorosulfonyl)phenoxyacetic acid 2-(tolylsulfonyl)ethyl ester (3.75 g, 8.67 mmol, 1.2 eq) as a powder. After the chlorosulfonyl compound was dissolved completely, about 1 g of the solid NaHCO₃ was added to the mixture. After all the solvent was removed, the oil was purified by column chromatography (silica gel, CH₂Cl₂ to 2% MeOH in CH₂Cl₂) to give 3.90 g (73%) of the mixture product, 2-tosylethyl 2-(4-(5-methoxy-2-((4-methoxy-3,5-dimethylpyridin-2-yl)methylsulfinyl)-1H-imidazo[4,5-b]pyridin-1-ylsulfonyl)phenoxy)acetate and 2-tosylethyl 2-(4-(5-methoxy-2-((4-methoxy-3,5-dimethylpyridin-2-yl)methylsulfinyl)-3H-imidazo[4,5-b]pyridin-3-ylsulfonyl)phenoxy)acetate, as off-white foam.

¹H NMR (400 MHz, CDCl₃) δ2.22 (s, 3H), 2.32 (s, 3H), 2.41 (s, 3H), 3.43 (t, 2H), 3.75 (s, 3H), 4.00 (s, 3H), 4.53 (m, 4H), 5.00 (dd, 2H), 6.88 (d, 1H), 6.96 (d, 2H), 7.35 (d, 2H), 7.76 (d, 2H), 8.02 (s, 1H), 8.14 (d, 2H), 8.20 (d, 1H).

The ester mixture of 2-tosylethyl 2-(4-(5-methoxy-2-((4-methoxy-3,5-dimethylpyridin-2-yl)methylsulfinyl)-1H-imidazo[4,5-b]pyridin-1-ylsulfonyl)phenoxy)acetate and 2-tosylethyl 2-(4-(5-methoxy-2-((4-methoxy-3,5-dimethylpyridin-2-yl)methylsulfinyl)-3H-imidazo[4,5-b]pyridin-3-ylsulfonyl)phenoxy)acetate (3.8 g, 5.1 mmol) was dissolved in 40 mL of CH₃CN, and then the solution of NaHCO₃ (475 mg, 5.6 mmol, 1.1 eq) in 20 mL of H₂O. The mixture was heated to 65-70° C. for 45 min.

Acetonitrile was evaporated under reduced pressure. Aq-layer was extracted with ethyl acetate (2×50 mL) for removing vinylsulfonyltoluene, the trace amount of tenatoprazole and the starting material. Aqueous layer was lyophilized to give lyophilized product. Methylene chloride (100 ml) was added to dissolve the product (Na salt), and filtered to remove NaHCO₃, and (p-sulfo)phenoxyacetic acid sodium salt, and dried under reduced pressure to yield 2.3 g (77%) of bright yellow solid, 1:1 mixture of sodium 2-(4-(5-methoxy-2-((4-methoxy-3,5-dimethylpyridin-2-yl)methylsulfinyl)-1H-imidazo[4,5-b]pyridin-1-ylsulfonyl)phenoxy)acetate (Compound 1) and sodium 2-(4-(5-methoxy-2-((4-methoxy-3,5-dimethylpyridin-2-yl)methyl sulfinyl)-3H-imidazo[4,5-b]pyridin-3-ylsulfonyl)phenoxy)acetate.

¹H NMR {400 MHz, D₂O:DMSO-d₆(3:1)} δ1.80 (s, 3H), 2.11 (s, 3H), 3.62 (s, 3H), 3.80 (s, 3H), 4.38 (s, 2H), 4.87 (dd, 2H), 6.87 (d, 1H), 6.99 (d, 2H), 7.78 (s, 1H), 7.93 (d, 2H), 8.15 (d, 1H).

1. A compound of Formula 1 or of Formula 2 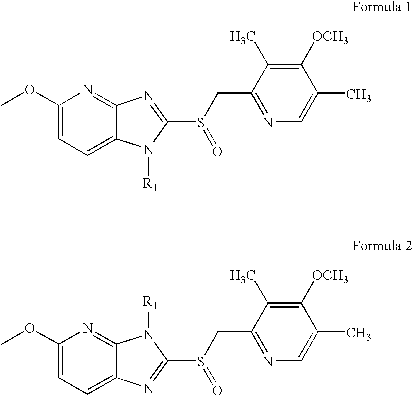
where R₁ is represented by Formula 3 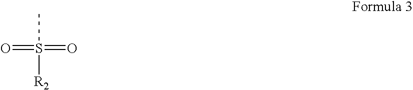
the dashed line represents the bond connecting the sulfur atom of Formula 3 to the nitrogen atom in the 1H-imidazo[4,5-b]pyridine nucleus;
R₂ is phenyl, naphthyl or heteroaryl having 1 to 3 heteroatoms independently selected from N, O and S, said phenyl, naphthyl or heteroaryl groups being unsubstituted or substituted with 1 to 5 R₃ groups;
R₃ is alkyl of 1 to 10 carbons, halogen substituted alkyl of 1 to 10 carbons, alkoxy having 1 to 10 carbons, halogen substituted alkoxy of 1 to 10 carbons, alkylthio having 1 to 10 carbons, halogen substituted alkylthio of 1 to 10 carbons, alkoxy carbonyl having 1 to 10 carbons, halogen substituted alkoxy carbonyl having 1 to 10 carbons, F, Cl, Br, I, NO₂, CN, OCOalkyl, NH₂, alkylamino and dialkylamino where in said OCOalkyl, alkylamino and dialkylamino groups each of said alkyl group has 1 to 10 carbons, or a pharmaceutically acceptable salt of said compound.

2. A compound in accordance with claim 1, having the structure of Formula 1, or a pharmaceutically acceptable salt of said compound.

3. A compound in accordance with claim 2, wherein R₂ is phenyl, or a pharmaceutically acceptable salt of said compound.

4. A compound in accordance with claim 1, having the structure of Formula 2, or a pharmaceutically acceptable salt of said compound.

5. A compound in accordance with claim 4, wherein R₂ is phenyl, or a pharmaceutically acceptable salt of said compound.

6. A compound of Formula 4 or of Formula 5 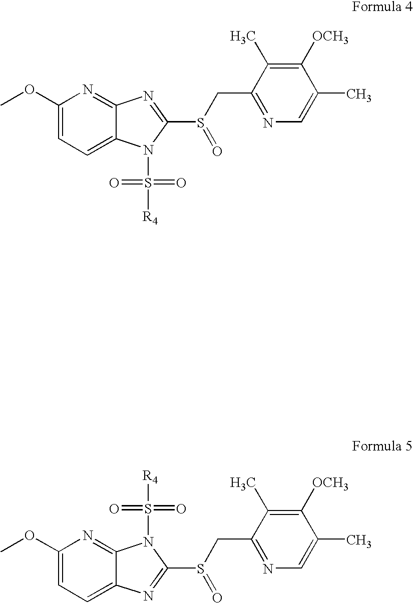
where R₄ represents the groups selected from Formulas (i) through (viii). 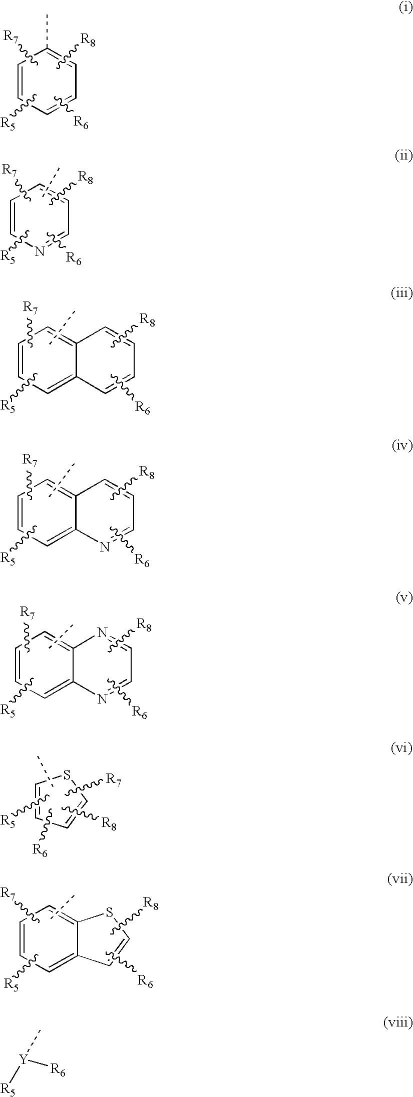
the dashed line represents the bond connecting the R₄ group with the SO₂ group;
Y is a straight chained or branch-chained disubstituted alkyl group of 1 to 8 carbons, or Y is N;
R₅ and R₆ independently are H, a straight chained or branch-chained di- or trisubstituted alkyl group of 1 to 12 carbons including 1 or two R₉ groups, or a straight chained or branch-chained saturated hydrocarbon skeleton having no more than 12 carbons including 1 or two R₉ groups and optionally further including one to three X groups where X is independently selected from the group consisting of —O—, —S—, —NR₁₀—, —NHCO—, —CONH—, —CONHCO—, —COO—, —OCO— and a disubstituted phenyl group which can optionally be substituted with one or two halogen atoms or with one or two R₇ groups; or the R₉ group is directly attached without an intervening R₅ or R₆ group to the aromatic or heteroaromatic ring or to the Y group of formulas (i) through (viii);
R₇ and R₈ independently are H, alkyl of 1 to 3 carbons, fluoroalkyl of 1 to 3 carbons, O-alkyl of 1 to 3 carbons, O-fluoroalkyl of 1 to 3 carbons, S-alkyl of 1 to 3 carbons, S-fluoroalkyl of 1 to 3 carbons;
R₉ is independently H, COOH or a tetrazole moiety;
R₁₀ is H or alkyl of 1 to 3 carbons;
with the provisos that
at least one the R₅ and R₆ groups is not H, and
at least one R₉ is not H and no more than two R₉ groups are COOH or tetrazole whereby the compound includes at least one but no more than two COOH or tetrazole groups;
when Y is —N then neither of the R₅ and R₆ groups is H,
or a pharmaceutically acceptable salt of said compound.

7. A compound in accordance with claim 6 where R₄ is selected from the group consisting of formulas (i), (ii), (iii) and (vi) or a pharmaceutically acceptable salt of said compound.

8. A compound in accordance with claim 7 where R₄ has formula (i), or a pharmaceutically acceptable salt of said compound.

9. A compound in accordance with claim 6 where R₉ is COOH, or a pharmaceutically acceptable salt of said compound.

10. A compound of the formula 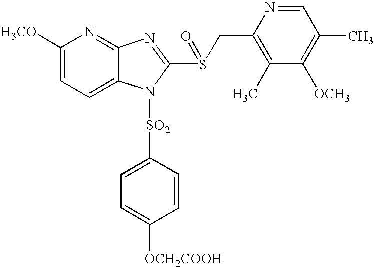
or a pharmaceutically acceptable salt of said compound.

11. A pharmaceutical composition comprising a pharmaceutically acceptable excipient and a compound in accordance with claim 1.

12. A pharmaceutical composition in accordance with claim 11 and having a compound of claim 3.

13. A pharmaceutical composition in accordance with claim 11 and having a compound of claim 5.

14. A pharmaceutical composition in accordance with claim 11 and having a compound of claim 6.

15. A pharmaceutical composition in accordance with claim 11 and having a compound of claim 7.

16. A pharmaceutical composition in accordance with claim 11 and having a compound of claim 8.

17. A pharmaceutical composition in accordance with claim 11 and having a compound of claim 9.

18. A pharmaceutical composition in accordance with claim 11 and having a compound of claim 10.

19. A pharmaceutical composition comprising a pharmaceutically acceptable excipient and a compound in accordance with claim 1 and a proton pump inhibitor drug selected from the group consisting of the compounds having the formulas (u), (v), (w), (x), (y) and (z). [chemical structure]
